(19) United States
(12) Patent Application Publication
(10) Pub. No.: US 2017/0320730 A1
(43) Pub. Date: Nov. 9, 2017

(54) INTEGRATION OF SYNGAS PRODUCTION FROM STEAM REFORMING AND DRY REFORMING

(71) Applicants: Aghaddin MAMEDOV, Sugar Land, TX (US); SABIC Global Technologies B.V., Bergen op Zoom (NL)

(72) Inventor: Aghaddin MAMEDOV, Sugar Land, TX (US)

(21) Appl. No.: 15/520,606

(22) PCT Filed: Oct. 23, 2015
(86) PCT No.: PCT/US2015/057039

§ 371 Date: Apr. 20, 2017

Related U.S. Application Data
(60) Provisional application No. 62/068,812, filed on Oct. 27, 2014.

(51) Int. Cl. C01B 3/38; C07C 1/04; B01J 23/83; C07C 29/151

(52) U.S. Cl. CPC C01B 3/382; C07C 29/1518; C07C 1/0485; B01J 23/83; C01B 2203/1241; C01B 2203/062; C01B 2203/061; C01B 2203/048; C01B 2203/0238; C01B 2203/0233; C01B 2203/141; C01B 2203/82; C07C 2523/83; C01B 2203/1058

(57) ABSTRACT

Processes for converting methane into an olefin and methanol are provided. The olefin can be ethylene. Certain exemplary processes can involve parallel use of both steam reforming of methane and oxidative dry reforming of methane to prepare syngas. The processes can further involve conversion of syngas to ethylene and to methanol.

### FIELD

The presently disclosed subject matter relates to processes and systems for converting methane into an olefin and methanol.

### BACKGROUND

Synthesis gas, also known as syngas, is a gas mixture containing hydrogen (H₂) and carbon monoxide (CO). Syngas can also include carbon dioxide (CO₂). Syngas is a chemical feedstock that can be used in numerous applications. For example, syngas can be used to prepare liquid hydrocarbons, including olefins (e.g., ethylene (C₂H₄)), via the Fischer-Tropsch process. Syngas can also be used to prepare methanol (CH₃OH).

Conversion of syngas to ethylene under Fischer-Tropsch conditions can proceed according to the following chemical equation:

2CO+4H₂→C₂H₄+2H₂O

Conversion of syngas to methanol can proceed according to the following chemical equation:

CO+2H₂→CH₃OH

Consequently, syngas with a molar ratio of hydrogen to carbon monoxide of 2:1 can be useful for the formation of ethylene and/or methanol. Use of syngas with a higher molar ratio of hydrogen to carbon monoxide (e.g., 3:1 or higher) can cause problems. For example, use of syngas with a high molar ratio of hydrogen to carbon monoxide in preparation of ethylene can reduce selectivity for ethylene and increase formation of undesired side products.

Syngas is commonly generated on large scale from methane (CH₄), e.g., through steam reforming processes or through oxidative reforming with oxygen (in the absence of carbon dioxide). Existing processes can suffer from drawbacks. For example, steam reforming processes can be affected by harmful coke formation. Steam reforming processes can also be highly endothermic and energy intensive. Oxidative reforming with oxygen can be highly exothermic and can consequently cause problematic exotherms.

An additional drawback with preparation of syngas via steam reforming and oxidative reforming with oxygen can be that certain reactions provide syngas with a molar ratio of hydrogen to carbon monoxide of approximately 3:1 or higher, greater than the 2:1 ratio ideal for formation of ethylene and for formation of methanol. Thus, there remains a need for improved processes for preparation of syngas from methane and improved processes for preparation of olefins (e.g., ethylene) and methanol from syngas.

### SUMMARY OF THE DISCLOSED SUBJECT MATTER

The presently disclosed subject matter provides processes for converting methane into an olefin (e.g., ethylene) and methanol.

In one embodiment, an exemplary process for converting methane into an olefin and methanol can include contacting methane, carbon dioxide, and oxygen with an oxidative dry reforming catalyst to provide an oxidative dry reforming product mixture. The oxidative dry reforming product mixture can include carbon monoxide, hydrogen, and water. The process can further include contacting methane and water with a steam reforming catalyst to provide a steam reforming product mixture that includes carbon monoxide and hydrogen.

In addition, the oxidative dry reforming product mixture can be put in contact with an olefin preparation catalyst to provide an olefin product mixture that includes an olefin and carbon monoxide. The process can further include separating carbon monoxide from the olefin product mixture to provide separated carbon monoxide, and the separated carbon monoxide can be combined with at least a portion of the steam reforming product mixture to provide a methanol preparation mixture. The process can further include contacting the methanol preparation mixture with a methanol preparation catalyst to provide methanol.

In certain embodiments, the oxidative dry reforming catalyst can include a solid support. The solid support can include at least one support, such as one or more of alumina, silica, and magnesia.

In certain embodiments, the oxidative dry reforming catalyst can include nickel. In certain embodiments, the oxidative dry reforming catalyst can include nickel in an amount between about 2% and about 15%, by weight, relative to the total weight of the catalyst.

In certain embodiments, the oxidative dry reforming catalyst can include a basic metal oxide. The basic metal oxide can include lanthanum(III) oxide.

In certain embodiments, the oxidative dry reforming catalyst can include a noble metal. The noble metal can include at least one noble metal, such as one or both of platinum and ruthenium. In certain embodiments, the oxidative dry reforming catalyst can include the noble metal in an amount between about 0.1% and about 2%, by weight, relative to the total weight of the catalyst.

In certain embodiments, the methanol preparation mixture can include hydrogen and carbon monoxide in a molar ratio of about 2:1.

In certain embodiments, contacting methane, carbon dioxide, and oxygen with the oxidative dry reforming catalyst, contacting methane and water with the steam reforming catalyst, contacting the oxidative dry reforming product mixture with the olefin preparation catalyst, and contacting the methanol preparation mixture with the methanol preparation catalyst can occur concurrently.

In certain embodiments, the olefin can include ethylene.

In one embodiment, an exemplary process for converting methane into ethylene and methanol can include contacting methane, carbon dioxide, and oxygen with an oxidative dry reforming catalyst to provide an oxidative dry reforming product mixture that includes carbon monoxide, hydrogen and water. The oxidative dry reforming catalyst can include nickel, a basic metal oxide, and a noble metal. The process can further include contacting methane and water with a steam reforming catalyst to provide a steam reforming product mixture that includes carbon monoxide and hydrogen. The process can additionally include contacting the oxidative dry reforming product mixture with an olefin preparation catalyst to provide an olefin product mixture that includes ethylene and carbon monoxide. The process can further include separating carbon monoxide from the olefin product mixture to provide separated carbon monoxide, and combining separated carbon monoxide with at least a portion of the steam reforming product mixture to provide a methanol preparation mixture. The process can further include contacting the methanol preparation mixture with a methanol preparation catalyst to provide methanol.

### BRIEF DESCRIPTION OF THE DRAWINGS

FIG. 1 is a schematic diagram showing an exemplary system that can be used in conjunction with processes for converting methane into an olefin and methanol in accordance with the presently disclosed subject matter.

### DETAILED DESCRIPTION

The presently disclosed subject matter provides processes for converting methane into an olefin (e.g., ethylene) and methanol. The presently disclosed processes can provide syngas with a molar ratio of hydrogen:carbon monoxide of about 2:1. The presently disclosed processes can involve parallel use of both steam reforming of methane and oxidative dry reforming of methane. The presently disclosed processes can have advantages over existing processes, as described below, including improved efficiency, reduced energy consumption, and reduced cost.

As used herein, the term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5%, and or up to 1% of a given value.

Steam reforming of methane is a process in which methane is reacted with water (steam) to provide carbon monoxide and hydrogen. Steam reforming can be summarized by the following chemical equation:

CH₄+H₂O→CO+3H₂. (1)

In certain embodiments, carbon monoxide formed in steam reforming processes can react with water to form carbon dioxide and hydrogen, according the following chemical equation:

CO+H₂O→CO₂+H₂ (2)

Steam reforming of methane can provide syngas with a molar ratio of hydrogen to carbon monoxide of approximately 3:1. In certain embodiments (e.g., when carbon monoxide reacts with water to form carbon dioxide and water), steam reforming of methane can provide syngas with a molar ratio of hydrogen to carbon monoxide greater than 3:1.

Oxidative dry reforming of methane is a process in which methane is reacted with carbon dioxide and oxygen to provide carbon monoxide, hydrogen, and water. Oxidative dry reforming can be summarized by the following chemical equation:

2CH₄+CO₂+O₂→3CO+3H₂+H₂O (3)

Oxidative dry reforming can accordingly generate syngas with a hydrogen:carbon monoxide ratio of approximately 1:1.

Processes for converting methane into an olefin (e.g., ethylene) and methanol of the presently disclosed subject matter can generally include contacting methane, carbon dioxide, and oxygen with an oxidative dry reforming catalyst to provide an oxidative dry reforming product mixture that includes carbon monoxide, hydrogen, and water. The processes can further include contacting methane and water with a steam reforming catalyst to provide a steam reforming product mixture that includes carbon monoxide and hydrogen. The processes can additionally include contacting the oxidative dry reforming product mixture with an olefin preparation catalyst to provide an olefin product mixture that includes an olefin and carbon monoxide. The processes can further include separating carbon monoxide from the olefin product mixture to provide separated carbon monoxide. The processes can additionally include combining separated carbon monoxide with at least a portion of the steam reforming product mixture to provide a methanol preparation mixture. The processes can further include contacting the methanol preparation mixture with a methanol preparation catalyst to provide methanol.

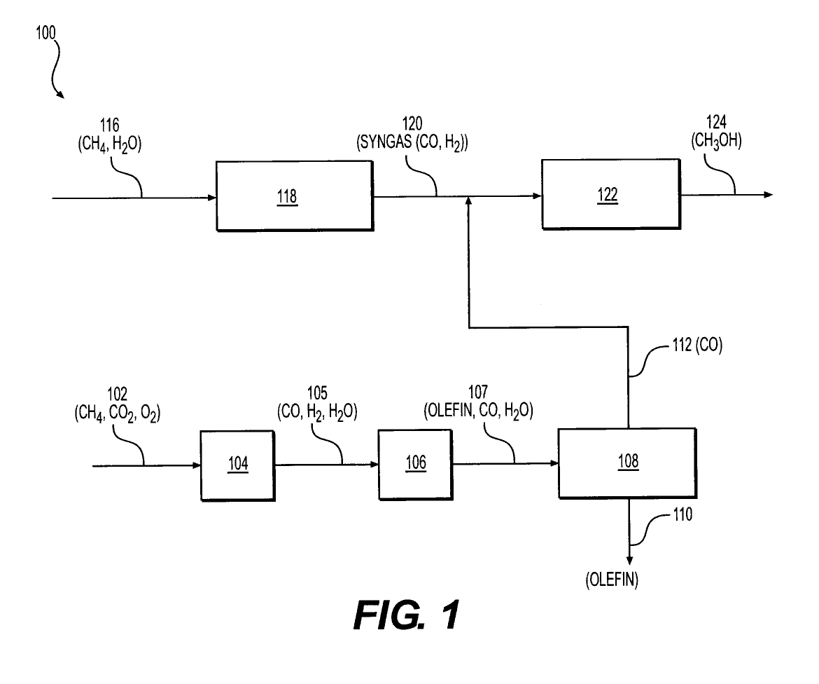

For the purpose of illustration and not limitation, FIG. 1 is a schematic representation of an exemplary system that can be used in conjunction with the processes of the presently disclosed subject matter. The system 100 can include an oxidative dry reforming reactor 104. The oxidative dry reforming reactor 104 can include an oxidative dry reforming catalyst. A stream 102 that contains methane, carbon dioxide, and oxygen can be fed into the reactor 104 and can be contacted with the oxidative dry reforming catalyst to provide an oxidative dry reforming product mixture that contains carbon monoxide, hydrogen, and water. The proportions of methane, carbon dioxide, and oxygen in the stream 102 can be varied. In certain embodiments, the molar ratio of methane:carbon dioxide:oxygen can be about 2:1:1. In certain embodiments, an excess of methane can be used. In certain embodiments, the stream 102 can include nitrogen (N₂). The oxidative dry reforming product mixture can be removed as a stream 105 from the reactor 104. In certain embodiments, the oxidative dry reforming product mixture can also include unreacted methane and/or carbon dioxide.

In certain embodiments, the stream 102 containing methane, carbon dioxide, and oxygen can be dry. That is, the reaction mixture stream 102 can be free of water. Use of a dry reaction mixture can help to reduce energy consumption.

The reactor 104 can be of various designs known in the art. In certain embodiments, the reactor can be a fixed bed plug flow reactor. In certain embodiments, the reactor can be a fluidized bed or riser-type reactor.

In certain embodiments, the oxidative dry reforming catalyst in the reactor 104 can be used as a bulk mixture. By way of non-limiting example, the reactor 104 can be packed with particles, granules, and/or pellets of catalyst.

In certain embodiments, the oxidative dry reforming catalyst in the reactor 104 can include can include a solid support. That is, the oxidative dry reforming catalyst can be solid-supported. In certain embodiments, the solid support can include various metal salts, metalloid oxides, and metal oxides, e.g., titania (titanium oxide), zirconia (zirconium oxide), silica (silicon oxide), alumina (aluminum oxide), magnesia (magnesium oxide), and magnesium chloride. In certain embodiments, the solid support can include alumina (Al₂O₃), silica (SiO₂), magnesia (MgO), or a combination thereof. In certain embodiments, the solid support can include lanthanum(III) oxide (La₂O₃).

In certain embodiments, the oxidative dry reforming catalyst can include nickel (Ni). The oxidative dry reforming catalyst can include one or more nickel oxides (e.g., NiO and/or Ni₂O₃). The oxidative dry reforming catalyst can include nickel metal (Ni(0)), e.g., nickel supported on a solid support. In certain embodiments, the oxidative dry reforming catalyst can include nickel in an amount between about 2% and about 15%, by weight, relative to the total weight of the catalyst. For example, when the oxidative dry reforming catalyst includes a solid support, the catalyst can include nickel in an amount between about 2% and about 15%, by weight, relative to the total weight of the catalyst, and the remainder of the catalyst can be solid support. In certain embodiments, the catalyst can include nickel in an amount of about 2%, relative to the total weight of the catalyst.

In certain embodiments, the oxidative dry reforming catalyst can include one or more metal oxides that is not a nickel oxide. By way of non-limiting example, suitable metal oxides can include chromium oxides (e.g., Cr₂O₃), manganese oxides (e.g., MnO, MnO₂, Mn₂O₃, or Mn₂O₇), copper oxides (e.g., CuO), tin oxides (e.g., SnO₂), lanthanum oxides (e.g., La₂O₃), cerium oxides (e.g., CeO₂), and tungsten oxides (e.g., WO₃). In certain embodiments, the catalyst can include oxides of two, three, four, or more different metals (elements).

Loading of the oxidative dry reforming catalyst can be proportional to the size of the reactor 104. By of way of non-limiting example, a reactor that includes a quartz tube of internal diameter 10 mm and length between about 0.5 inches and 3 inches can be loaded with about 0.5 mL of the oxidative dry reforming catalyst. The oxidative dry reforming catalyst can be diluted with quartz particles. The size of the catalyst and quartz particles can be in a range from about 20 to about 50 mesh.

In certain embodiments, the oxidative dry reforming catalyst in the reactor 104 can include a basic metal oxide. Basic metal oxides are metal oxides with basic properties. For example, basic metal oxides include metal oxides that can react with an acid to form a salt and water. In certain embodiments, the basic metal oxide can include at least one basic metal oxide such as lithium oxides (e.g., Li₂O), sodium oxides (e.g., Na₂O), potassium oxides (e.g., K₂O), calcium oxides (e.g., CaO), strontium oxides (e.g., SrO), barium oxides (e.g., BaO), and lanthanum oxides (e.g., La₂O₃). In certain embodiments, the basic metal oxide can be lanthanum(III) oxide (La₂O₃). Lanthanum(III) oxide can have mildly basic character.

In certain embodiments, the oxidative dry reforming catalyst in the reactor 104 can include one or more basic metal oxides in an amount between about 1% and about 5%, by weight, relative to the total weight of the catalyst. For example, when the catalyst includes a solid support and nickel, the catalyst can include the basic metal oxide in an amount between about 1% and about 5%, by weight, relative to the total weight of the catalyst, and the remainder of the catalyst can be solid support and the nickel species.

In certain embodiments, the oxidative dry reforming catalyst in the reactor 104 can include one or more noble metals (e.g., Ru, Rh, Ph, Ag, Os, Ir, Pt, or Au). The noble metal can be platinum (Pt), ruthenium (Ru), or a combination thereof. In certain embodiments, the oxidative dry reforming catalyst can include one or more noble metals in an amount between about 0.1% and 2%, by weight, relative to the total weight of the catalyst.

Oxidative dry reforming catalysts can be prepared by various methods known in the art. By way of non-limiting example, oxidative dry reforming catalysts can be prepared by precipitation, e.g., by precipitation from corresponding nitrate salts by treatment with NH₄OH. Oxidative dry reforming catalysts can also be prepared by mixing of salts and/or by calcination.

In certain embodiments, the temperature in the oxidative dry reforming reactor 104 can be between about 550° C. and about 950° C. In certain embodiments, the reaction mixture can be contacted with the catalyst at a temperature between about 650° C. and about 720° C. In certain embodiments, the reaction mixture can be contacted with the catalyst at a temperature between about 800° C. and about 850° C.

In certain embodiments, the oxidative dry reforming reactor 104 can be operated at atmospheric pressure. In other embodiments, the reactor 104 can be operated at elevated pressure. For example, the reactor 104 can be operated at a pressure between atmospheric pressure and about 30 bar, e.g., in a range between about 20 bar and about 25 bar.

In certain embodiments, the oxidative dry reforming reactor 104 can have a gas hourly space velocity (GHSV) of between about 2,000 h⁻¹ and about 20,000 h⁻¹, e.g., between about 5,000 h⁻¹ and about 10,000 h⁻¹. By way of non-limiting example, the GHSV of the reactor 104 can be about 2,000 h⁻¹ when the reactor includes a Pt-based catalyst. The GHSV of the reactor 104 can be in a range from about 1,900 to about 3,600 h⁻¹ when the reactor includes a catalyst based on Ni and La₂O₃.

An oxidative dry reforming product mixture containing carbon monoxide, hydrogen, and water can be removed as a stream 105 from the reactor 104. The oxidative dry reforming product mixture can contain hydrogen and carbon monoxide in a molar ratio of about 1:1, as described above. The stream 105 of oxidative dry reforming product mixture can be fed into an olefin preparation reactor 106. In certain embodiments, the stream 105 of oxidative dry reforming product mixture can be dried before being fed into the olefin preparation reactor 106, e.g., by distillation and/or by passage through a drying agent (e.g., calcium chloride). The olefin preparation reactor 106 can include an olefin preparation catalyst. Contacting the oxidative dry reforming product mixture (a syngas mixture) with the olefin preparation catalyst can induce a Fischer-Tropsch reaction to form an olefin. That is, contacting the oxidative dry reforming product mixture can provide an olefin product mixture that includes an olefin and carbon monoxide. The olefin product mixture can be removed as a stream 107 from the olefin preparation reactor 106.

The olefin preparation catalyst in the olefin preparation reactor 106 can be an olefin preparation catalyst known in the art. For example, the olefin preparation catalyst can include iron (Fe), manganese (Mn), or a combination thereof. By way of non-limiting example, the olefin preparation catalyst can include one or more of a Fe—Mn/Al₂O₃ catalyst, a Co—Mn/Al₂O₃ catalyst, a Co—Mn—K/Al₂O₃ catalyst, and an iron-based catalyst. The olefin preparation catalyst can include one or more alkali metals. The olefin preparation reactor 106 can be operated at conditions known in the art, e.g., a temperature between about 400° C. and about 450° C., a pressure between about 20 bar and about 50 bar, and a contact time of about 1 second to about 3 seconds.

In certain embodiments, the olefin formed in the olefin preparation reactor 106 can include ethylene. The olefin formed in the olefin preparation reactor 106 can also include one or more of propylene, butene (various isomers), and pentene (various isomers).

The olefin product mixture stream 107 containing an olefin (e.g., ethylene), carbon monoxide, and water can be fed to a separation unit 108. The separation unit 108 can separate and remove water from the product mixture. In certain embodiments, separating water from the product mixture can include cooling the product mixture. In other words, the separation unit 108 can cool the product mixture to condense water. By way of non-limiting example, the temperature within the separation unit 108 can be between about 5° C. and about 10° C. and the pressure can be between about 1 bar and 20 bar.

In certain embodiments, the separation unit 108 can separate carbon monoxide and the olefin from the product mixture. The separation unit 108 can separate various components by distillation. An olefin stream 110 and a carbon monoxide stream 112 can be removed from the separation unit 108. The olefin (e.g., ethylene) can be isolated as a product of the process. Unreacted methane and/or carbon dioxide can also be recovered from the separation unit 108 and optionally recycled.

The system 100 can include a steam reforming reactor 118. The steam reforming reactor 118 can include a steam reforming catalyst. A stream 116 containing methane and water can be fed to the reactor 118. In certain embodiments, the stream 116 can contain methane and water in a molar ratio between about 1:1 and about 3:1, e.g., about 1:1, about 2:1, or about 3:1. Contacting methane and water with the steam reforming catalyst can provide a steam reforming product mixture that contains carbon monoxide and hydrogen (i.e., syngas). The steam reforming product mixture can contain hydrogen and carbon monoxide in a molar ratio of about 3:1 or greater, as described above, e.g., about 3:1, about 4:1, about 5:1, about 6:1, or higher than 6:1. The steam reforming product mixture can be removed as a stream 120 from the steam reforming reactor 118. In certain embodiments, the stream reforming product mixture 120 can be dried before further use, e.g., by condensation, distillation, and/or by passage through a drying agent (e.g., calcium chloride).

The steam reforming catalyst in the steam reforming reactor 118 can be a methane steam reforming catalyst known in the art. For example, the steam reforming catalyst can include nickel (Ni). In certain embodiments, the steam reforming catalyst can include one or more alkaline earth elements and can be solid supported (e.g., on Al₂O₃). By way of non-limiting example, the temperature within the steam reforming reactor 118 can be between about 850° C. and 1000° C. When the steam reforming reactor 118 is operated under adiabatic conditions, the temperature can be greater than 1000° C. The pressure within the steam reforming reactor 118 can be between 25 bar and 40 bar.

The carbon monoxide stream 112 removed from the separation unit 108 can be combined with at least a portion of the steam reforming product mixture stream 120. As described above, the steam reforming product mixture stream 120 can include hydrogen and carbon monoxide in a molar ratio of about 3:1 or greater. Upon mixing with the carbon monoxide stream 112, the molar ratio of hydrogen to carbon monoxide can decrease. The steam reforming product mixture stream 120 and carbon monoxide stream 112 can be mixed in various proportions to provide a combined syngas stream that includes hydrogen and carbon monoxide in a molar ratio between about 1:1 and about 3:1. For example, the combined syngas stream can include hydrogen and carbon monoxide in a molar ratio of about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, or about 3:1. In certain embodiments, the combined syngas stream can include hydrogen and carbon monoxide in a molar ratio of about 2:1.

The combined syngas stream prepared by combining separated carbon monoxide from the separation unit 108 and at least a portion of the steam reforming product mixture can be described as a methanol preparation mixture. The combined syngas stream (methanol preparation mixture) can be fed to a methanol preparation reactor 122. The methanol preparation reactor 122 can include a methanol preparation catalyst. Contacting the combined syngas stream (methanol preparation mixture) with the methanol preparation catalyst can provide methanol. A methanol stream 124 can be removed from the methanol preparation reactor 122. Methanol can be collected as a product.

The methanol preparation catalyst in the methanol preparation reactor 122 can be a methanol preparation catalyst known in the art. For example, the methanol preparation catalyst can include copper (Cu), zinc (Zn), or a mixture thereof. The methanol preparation catalyst can include a Cu—Zn—O catalyst. By way of non-limiting example, the methanol preparation catalyst can include copper and nickel supported on alumina. The methanol preparation catalyst can include Ga, Zr, and/or Ce. Methanol preparation catalysts can be prepared by various methods known in the art, e.g., co-precipitation from nitrate salts. The temperature of the methanol preparation reactor 122 can be between about 230° C. and about 250° C. The pressure in the methanol preparation reactor 122 can be between about 30 bar and about 50 bar. The gas hourly space velocity (GHSV) of the methanol preparation reactor 122 can be between about 10,000 and about 12,000 h⁻¹. By way of non-limiting example, conversion of CO in the methanol preparation reactor 122 can be less than 100%, e.g., about 30%, and unreacted syngas can be recycled.

The processes of the presently disclosed subject matter can have advantages over certain processes for converting methane into an olefin and methanol. Because the methane, carbon dioxide, and oxygen stream 102 can be a dry mixture (i.e., free of water), the oxidative dry reforming reaction can be free of coke formation. That is, there can be no coke formation in the oxidative dry reforming reactor 104 or in downstream equipment. An absence of coke formation can obviate the need for costly and inefficient regeneration of catalysts due to buildup of coke.

An additional advantage of the presently disclosed subject matter can be the use of oxidative dry reforming for conversion of methane to syngas, rather than exclusive use of steam reforming. Whereas steam reforming is highly endothermic (and consequently highly energy intensive), oxidative dry reforming is only mildly exothermic, which can reduce energy consumption and facilitate control of heat released by the reaction, reducing risk of exotherms.

### EXAMPLES

The following example is provided by way of illustration and not by way of limitation.

### Example 1

An exemplary oxidative dry reforming reaction of methane was conducted to prepare syngas. A feed that contained 28.4% methane, 17.4% carbon dioxide, 11% oxygen, and 42.8% nitrogen (by mole) was fed into an oxidative dry reforming reactor. The oxidative dry reforming catalyst was 0.5 mL (0.75 g) of a Ni/La₂O₃ catalyst containing 2% Ni (by weight) on La₂O₃. The reaction was conducted at atmospheric pressure. The GHSV was 4,800 h⁻¹. Various runs were conducted at different temperatures, and the composition of the syngas product formed as well as the percent conversion of methane and carbon dioxide were measured.

The composition of the syngas formed is presented in Table 1.

TABLE 1

| | | | | | | | | Conversion | |
|---|---|---|---|---|---|---|---|---|---|
| Run | Temp. | Product composition, mole % | | | | | | (%) | |
| # | (° C.) | CO | H₂ | CH₄ | CO₂ | N₂ | O₂ | CH₄ | CO₂ |
| 1 | 660 | 17.4 | 31.2 | 9.7 | 4.88 | 35.4 | 0.4 | 67.7 | 65.5 |
| 2 | 660 | 16.5 | 32.7 | 10.7 | 6.01 | 37.8 | 0.4 | 66.4 | 62.3 |
| 3 | 685 | 18.7 | 34.5 | 8.0 | 3.46 | 34.4 | 0.43 | 72.6 | 84.9 |
| 4 | 710 | 22.7 | 34.6 | 6.0 | 3.05 | 33.2 | 0.43 | 72.7 | 86.1 |

Although the presently disclosed subject matter and its advantages have been described in detail, it should be understood that various changes, substitutions and alterations can be made herein without departing from the spirit and scope of the disclosed subject matter as defined by the appended claims. Moreover, the scope of the disclosed subject matter is not intended to be limited to the particular embodiments described in the specification. Accordingly, the appended claims are intended to include within their scope such alternatives.

What is claimed is:

1. A process for converting methane into an olefin and methanol, comprising:
a. contacting methane, carbon dioxide, and oxygen with an oxidative dry reforming catalyst to provide an oxidative dry reforming product mixture comprising carbon monoxide, hydrogen, and water;
b. contacting methane and water with a steam reforming catalyst to provide a steam reforming product mixture comprising carbon monoxide and hydrogen;
c. contacting the oxidative dry reforming product mixture with an olefin preparation catalyst to provide an olefin product mixture comprising an olefin and carbon monoxide;
d. separating carbon monoxide from the olefin product mixture to provide separated carbon monoxide;
e. combining separated carbon monoxide with at least a portion of the steam reforming product mixture to provide a methanol preparation mixture; and
f. contacting the methanol preparation mixture with a methanol preparation catalyst to provide methanol.

2. The process of claim 1, wherein the oxidative dry reforming catalyst comprises a solid support.

3. The process of claim 2, wherein the solid support comprises at least one support selected from the group consisting of alumina, silica, and magnesia.

4. The process of claim 1, wherein the oxidative dry reforming catalyst comprises nickel.

5. The process of claim 4, wherein the oxidative dry reforming catalyst comprises nickel in an amount between about 2% and about 15%, by weight, relative to the total weight of the catalyst.

6. The process of claim 4, wherein the oxidative dry reforming catalyst comprises a basic metal oxide.

7. The process of claim 6, wherein the basic metal oxide comprises lanthanum(III) oxide.

8. The process of claim 4, wherein the oxidative dry reforming catalyst comprises a noble metal.

9. The process of claim 8, wherein the noble metal comprises at least one noble metal selected from the group consisting of platinum and ruthenium.

10. The process of claim 8, wherein the oxidative dry reforming catalyst comprises the noble metal in an amount between about 0.1% and about 2%, by weight, relative to the total weight of the catalyst.

11. The process of claim 1, wherein the methanol preparation mixture comprises hydrogen and carbon monoxide in a molar ratio of about 2:1.

12. The process of claim 1, wherein contacting methane, carbon dioxide, and oxygen with the oxidative dry reforming catalyst, contacting methane and water with the steam reforming catalyst, contacting the oxidative dry reforming product mixture with the olefin preparation catalyst, and contacting the methanol preparation mixture with the methanol preparation catalyst occur concurrently.

13. The process of claim 1, wherein the olefin comprises ethylene.

14. A process for converting methane into ethylene and methanol, comprising:
a. contacting methane, carbon dioxide, and oxygen with an oxidative dry reforming catalyst to provide an oxidative dry reforming product mixture comprising carbon monoxide, hydrogen, and water, wherein the oxidative dry reforming catalyst comprises nickel, a basic metal oxide, and a noble metal;
b. contacting methane and water with a steam reforming catalyst to provide a steam reforming product mixture comprising carbon monoxide and hydrogen;
c. contacting the oxidative dry reforming product mixture with an olefin preparation catalyst to provide an olefin product mixture comprising ethylene and carbon monoxide;
d. separating carbon monoxide from the olefin product mixture to provide separated carbon monoxide;
e. combining separated carbon monoxide with at least a portion of the steam reforming product mixture to provide a methanol preparation mixture; and
f. contacting the methanol preparation mixture with a methanol preparation catalyst to provide methanol.
